(19) United States
(12) Patent Application Publication
(10) Pub. No.: US 2005/0251185 A1
(43) Pub. Date: Nov. 10, 2005

(54) Microkeratome and surgical blade for this

(76) Inventor: Detlev Paul Gebauer, Tiefenbronn (DE)

Correspondence Address: BUTZEL LONG, 350 SOUTH MAIN STREET, SUITE 300, ANN ARBOR, MI, 48104, US

(21) Appl. No.: 10/842,051

(22) Filed: May 7, 2004

(51) Int. Cl. A61F 9/00

(52) U.S. Cl. 606/166

(57) ABSTRACT

The invention relates to a microkeratome (1) for use in a LASEK procedure as well as to a knife (3) with a blade (6) for a microkeratome, said blade (6) having a defined bluntness. In order to avoid loosening of the epithelium with an alcoholic solution in the LASEK procedure, the epithelium is sliced away to leave a remaining hinge, this being achieved by means of the microkeratome (1) into which the knife (3) is inserted. The blade (6) of the knife (3) has a defined bluntness to prevent Bowman's membrane from being injured by the cut in the epithelium. It comprises a cutting-edge angle (β) ranging from 28 degrees to 35 degrees, a clearance angle (α) ranging from 0 degrees to 4 degrees, and a blade radius (R) ranging from 150 nm to 800 nm.

The present invention relates to a microkeratome and to a surgical blade for a microkeratome. In particular, the invention relates to a microkeratome and an associated blade which, although used according to the LASIK procedure, comprises the advantages of the LASEK procedure without assuming the drawbacks thereof. The LASIK and LASEK procedures will be briefly illustrated below.

What is called the LASIK procedure (Laser-ASsisted In-situ Keratomileusis) is a method for surgical eye operation to correct an ametropia of the eye by means of an excimer laser, this procedure being the most frequently used at present. According to the LASIK procedure, a corneal lamella that is approx. 150 to 160 μm thick is sliced away from the cornea of the eye. This is achieved by means of a microkeratome (special blade). However, the corneal lamella is not sliced away completely, but is still connected to the remaining cornea via a small residual piece. The residual piece that is not sliced away serves as a hinge of sorts from which the corneal lamella that has been sliced away is folded away to one side. Subsequently, the exposed tissue is reshaped as desired by means of a computer-controlled excimer laser, thus correcting the ametropia. Thereafter, the corneal lamella is folded back, closing the tissue treated. Thereby, sutures are unnecessary, rapid healing is supported and the formation of scars is prevented.

As an alternative thereto, what is called the LASEK procedure (Laser-Assisted SubEpithelial Keratomileusis) has been established, this procedure having being used more and more frequently in the recent past.

According to the LASEK procedure, only the uppermost, approx. 55 μm thick layer of the cornea of the eye, which is called the epithelium, is removed from the cornea. The underlying stromal tissue remains uninjured. This is achieved by means of a special device, called microtrephine, which comprises a circular knife with a cutting blade within a range of approx. 270 degrees. The microtrephine is placed on the eye, penetrates in the epithelium down to a depth of approx. 55 μm and is then repeatedly turned back and forth by approx. 10 degrees. As a result, a circular incision is cut into the epithelium within a range of approx. 280 degrees. In a similar manner as in the LASIK procedure, the remaining uncut area of approx. 80 degrees serves as a hinge of sorts to which the epithelium that was cut is attached. To make the cut, the circular knife is guided in a cylinder that is placed on the eye. After the cut, a diluted alcoholic solution is dripped into a cylinder placed on the eye so that the alcoholic solution comes into contact with the epithelium that was cut. The alcoholic solution serves to loosen the tissue. After approx. 30 seconds, the alcoholic solution is sucked off and the incised epithelium is pushed to the side of the hinge by means of a spatula. After these preparatory measures, the actual laser treatment on the exposed tissue is performed in the same manner as in the LASIK procedure described above.

Drawbacks of the Present LASEK Procedure

The use of alcohol is required for loosening the incised epithelium and for being able to push it aside. The use of alcohol, however, is difficult for the following reasons: In the first place, the alcohol must, under no circumstances, come into contact with the connective tissue adjacent to the incised epithelium during the operation because this would inflict great pain on the patient. In the second place, alcohol is a neurotoxin that is basically considered to be harmful. It is true that, at present, it is not entirely clear how deep the alcohol penetrates in the tissue and what is the damage resulting therefrom; but the harmful effect of alcohol is taken for granted. In the third place, there is the not inconsiderable risk that the 30-second exposure period of the alcohol on the tissue fails to be correctly observed during the operation.

It is also to disadvantage that the operating surgeon must have great skill in pushing aside the circularly incised epithelium and that the risk of the epithelium becoming damaged or unusable during the operation is not inconsiderable.

A device for scraping off the epithelium, having a large radius ranging from 2 to 13 μm and used to remove the epithelium without the use of an alcoholic solution, is known from U.S. 2003/0018348 A1.

The present invention aims at obviating the drawbacks described above, i.e. to enable surgery according to the LASEK procedure, that does not require great skill and facilitates clean separation of the epithelium from Bowman's membrane without any alcohol.

This problem is solved by a knife according to claim 1, by a microkeratome according to claim 5, and a use of a microkeratome according to claim 6.

Advantageous further developments are subjects of the subordinate claims.

In the modified LASEK procedure, it is not necessary to cut a circular incision into the epithelium by means of the microtrephine nor must the epithelium be pushed aside with a spatula. On the contrary, the microkeratome according to the invention and the blade applied therein can be used to perform both the LASEK procedure and the LASIK procedure, said blade having a defined bluntness. In other words, the epithelium is not loosened with alcohol, but is cleanly sliced away by means of the microkeratome, and a hinge serving to readjust the corneal lamella that was sliced away is also left. As compared with the depth of cut in the LASIK procedure, which is approx. 150 to 160 μm, the depth of cut here is considerably less, i.e. only approx. 55 μm, similar to the LASEK procedure. As a result, what is called Bowman's membrane underneath the epithelium as well as the underlying stromal tissue remain uninjured.

Only with the blade geometry that is defined according to the invention is it possible to cleanly penetrate the epithelium and to advance to Bowman's membrane without damaging the latter. A blade that is too sharp would also penetrate Bowman's membrane and cut into the stromal tissue; however, this must be avoided. A blade that is too blunt would not be able to separate the epithelium cleanly from Bowman's membrane so that, possibly, cells of the epithelium may remain on Bowman's membrane, this having to be avoided as well.

The knife blade having a defined bluntness can be produced from a cheap material, such as stainless steel, at low cost. Since it is possible to treat stainless steel with the usual grinding methods, the grinding procedure is simple and cost-effective. Hence, the knife blade can be used as a disposable blade, so that the knife blade is thrown away after having been used. As a result, it is, to advantage, no longer necessary to subject the knife blade to expensive cleaning and the risk of a transmission of germs, etc. is, advantageously, eliminated by this single use. In particular, the transmission of proteins, the so-called prions, which are held responsible for transmission of the Creutzfeldt-Jacob disease, can be eliminated. This is particularly worth mentioning because the prions can survive even if having been steam-sterilized in the autoclave in the usual manner.

Since the LASEK procedure can be performed according to the LASIK procedure by means of the blade according to the invention, the microkeratome that has, so far, been used for the LASIK procedure can be used for this purpose. In other words, the microkeratome can be used both for the present LASIK procedure and the LASEK procedure. It is only necessary to exchange the knives. As a result of this dual utilization, considerable cost can be saved.

Moreover, the microkeratome renders the operation much more easier, because the cut is made automatically to a large extent, so that great skill and experience are not required.

A presently preferred embodiment of the invention will be illustrated in more detail below with reference being made to the enclosed figures, in which:

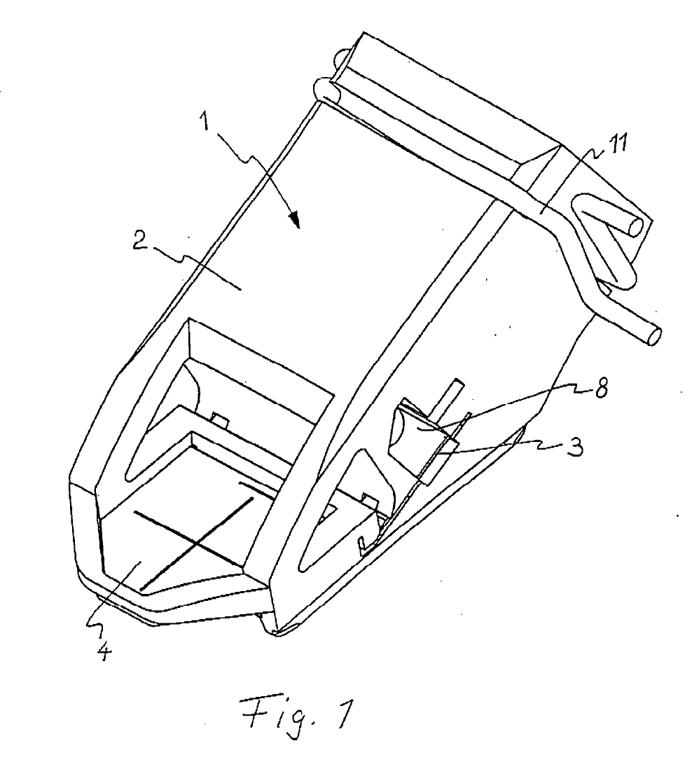

FIG. 1 is a perspective view of a microkeratome;

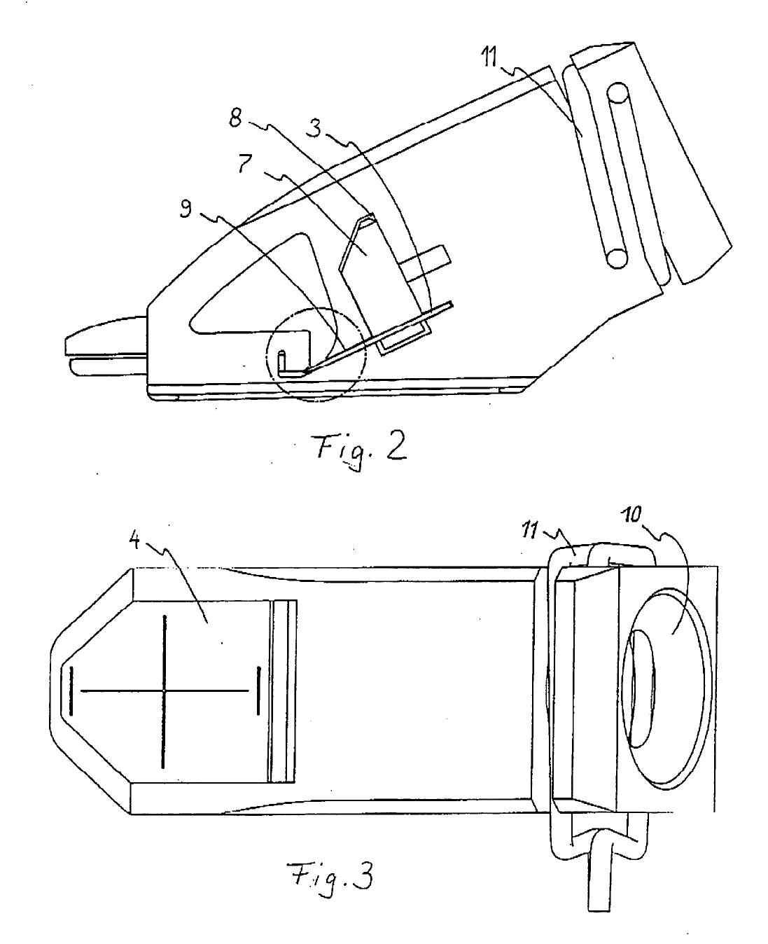

FIG. 2 is a lateral view of the microkeratome according to FIG. 1;

FIG. 3 is a top view of the microkeratome according to FIG. 1;

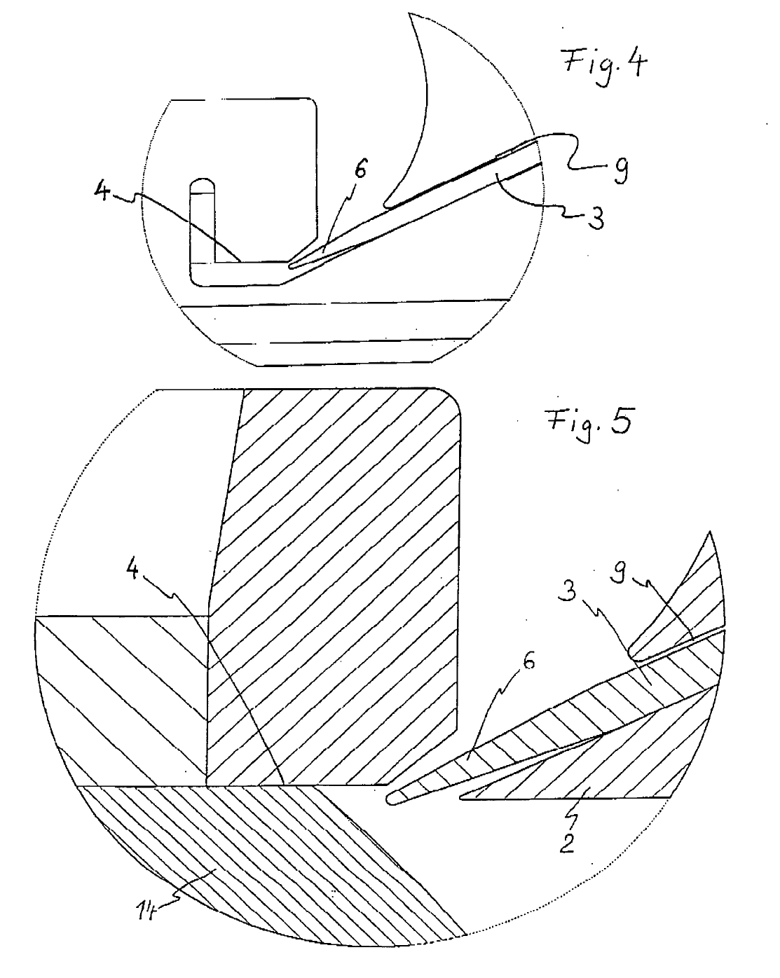

FIG. 4 is a magnified presentation of a circular sector from FIG. 2;

FIG. 5 is a magnified partial view of the surgical blade and its cutting geometry with indicated cornea and applanation surface; and

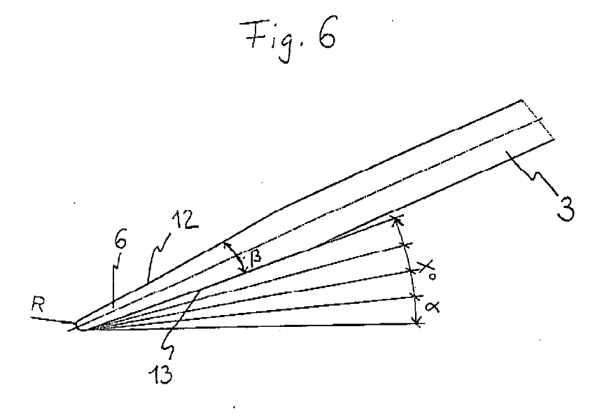

FIG. 6 is a magnified partial view of the surgical blade.

FIG. 1 shows a microkeratome 1 as it is usually also used for the present LASIK procedure. Since a microkeratome is known as such, only the essential features that are of relevance in connection with the present invention will be considered below. As for the rest, reference is, for example, made to EP-A-0 873 735 A1 with regard to the structure of a microkeratome. The microkeratome consists of a cutting head 2 that comprises a surgical blade or knife 3. At its forward end, the cutting head 2 is provided with an applanation surface 4 comprising a transparent glass with cross-hairs. The applanation surface 4 is intended to be placed on the cornea to be cut (cornea 14 in FIG. 5), wherein the cutting head 2 is fitted on a suction ring (not shown) that is fixed on the eye. The applanation surface 4 permits to apply a specific pressure to the cornea of the eye, this having a positive effect on the cut with the knife 3.

According to FIG. 2, a holding opening 8 is provided laterally in the cutting head 2, with a knife holder 7 being inserted into said holding opening 8. The knife holder 7 contains a knife 3. In the cutting head 2, a gap 9 serving to accept the knife 3 that is held in the knife holder 7 extends from the holding opening 8 towards the applanation surface 4.

As shown in FIG. 3, a coupling opening 10 is arranged opposite to the applanation surface 4, as seen in the longitudinal direction of the cutting head 2, wherein a motor drive that is connected to said coupling opening 10 is used to drive the knife 3, because the knife, like a shearing blade of a razor, is moved to and fro during cutting operation, in transverse direction to the cutting direction and in parallel to the blade, that is from left to right and back as seen from the front in FIG. 1. At the outer perimeter of the cutting head 2, a locking mechanism 11 is provided within the area of the coupling opening 10, said locking mechanism serving to fix the motor drive in the coupling opening. Best results are achieved with an oscillation frequency ranging from 3,000 to 10,000 rpm. Moreover, the movement of the knife 3 in the microkeratome is automatically made in cutting direction, wherein the optimum feed rate ranges from 0.5 to 1.55 mm/sec.

FIG. 4 shows a magnified circular sector from FIG. 2. The knife 3 extends through the gap 9 of the cutting head 2 towards the applanation surface 4. Therein, the blade 6 of the knife 3 projects to a point a little below the applanation surface 4 in the manner of a plane, so that a cut can be made at a depth of approx. 55 μm.

According to FIG. 5, the applanation surface 4 presses on the cornea 14 of the eye to be treated to flatten said cornea 14 before the blade 6 of the knife 3 penetrates in the epithelium.

The geometry of the blade 6 is illustrated in more detail below, with reference being made to FIG. 6. The blade 6 comprises an upper cutting surface 12 and a lower cutting surface 13. The clearance angle a is the angle between the lower cutting surface 12 and an imaginary horizontal line. The clearance angle is approx. 4 degrees in the present embodiment. However, the clearance angle can vary within a range from 0 to 4 degrees. The cutting-edge angle β is the angle between the upper and lower cutting surfaces 12, 13. It is approx. 30 degrees in the present embodiment. However, the clearance angle can vary within a range from 28 to 35 degrees. The blade radius is the radius of the tip of the blade. It is approx. 450 nm in the present embodiment. However, the blade radius can vary within a range from 150 to 800 nm.

While the cut is made, the blade 6 penetrates the epithelium down to Bowman's membrane, i.e. to a depth of approx. 55 μm. Since the cells of Bowman's membrane are somewhat more stable than the epithelium, the blade 6, owing to its defined bluntness, does not penetrate Bowman's membrane, but presses against Bowman's membrane without injuring it. As a result, the epithelium is pushed along Bowman's membrane while the cutting movement is being made. This procedure is relatively simple and does not require great skill.

Similarly to the present LASIK procedure, the epithelium is not sliced away completely, but a part thereof is left as a hinge, wherein the tissue that was pushed aside is folded around said hinge. Subsequently, the exposed tissue is treated with the help of an excimer laser, in order to correct the ametropia. Thereafter, the epithelium that was cut and pushed aside is pushed back onto the treated tissue.

1. Knife (3) for a microkeratome comprising a blade with a cutting-edge angle (β) ranging from approx. 28 degrees to approx. 35 degrees, with a clearance angle (α) ranging from approx. 0 degrees to approx. 4 degrees, and a blade radius (R) ranging from approx. 150 nm to approx. 800 nm.

2. Knife according to claim 1, wherein the blade radius, preferably, ranges from approx. 200 nm to approx. 600 nm.

3. Knife according to the preceding claim, wherein the blade radius, furthermore preferably, ranges from approx. 250 nm to approx. 500 nm.
